(19) United States
(12) Patent Application Publication
(10) Pub. No.: US 2022/0331342 A1
(43) Pub. Date: Oct. 20, 2022

(54) TREATMENT OF TRAUMATIC ENCEPHALOPATHY BY FIBROBLASTS AND THERAPEUTIC ADJUVANTS

(71) Applicant: FIGENE, LLC, Houston, TX (US)

(72) Inventors: Thomas ICHIM, San Diego, CA (US); Pete O'HEERON, Houston, TX (US)

(21) Appl. No.: 17/753,487

(22) PCT Filed: Sep. 9, 2020
(86) PCT No.: PCT/US2020/049990

§ 371 Date: Mar. 4, 2022

Related U.S. Application Data
(60) Provisional application No. 62/897,429, filed on Sep. 9, 2019.

(51) Int. Cl. A61K 31/65; A61K 31/155; A61K 45/06

(52) U.S. Cl. CPC A61K 31/65; A61K 31/155; A61K 45/06

(57) ABSTRACT

Embodiments of the disclosure include methods and compositions for treating neurological disorders by stimulating regenerative and anti-inflammatory activity of fibroblasts. In specific embodiments, fibroblasts are administered to an individual with one or more inhibitors of NFkappaB, including minocycline and/or analogues thereof. In specific cases, methods are utilized herein to treat or prevent central nervous system injury, such as chronic injuries including chronic traumatic encephalopathy.

The present application claims priority to U.S. Provisional Application Ser. No. 62/897,429, filed Sep. 9, 2019, which is incorporated by reference herein in its entirety.

### TECHNICAL FIELD

Embodiments of the disclosure encompass at least the fields of cell biology, molecular biology, neurology, physiology, biochemistry, immunology, and medicine.

### BACKGROUND

While numerous pathologies are the result of inflammation, it is known that inflammation is the body's reaction to injury and infection. Major events involved in inflammatory processes include increased blood supply to the injured or infected area; increased capillary permeability enabled by retraction of endothelial cells; and migration of leukocytes out of the capillaries and into the surrounding tissue. White cells can exit circulation in part due to increased capillary permeability allows larger molecules and cells to cross the endothelium that are not ordinarily capable of doing so, thereby allowing soluble mediators of immunity and leukocytes to reach the injured or infected site. Leukocytes, primarily neutrophil polymorphs (also known as polymorphonuclear leukocytes, neutrophils or PMNS) and macrophages, migrate to the injured site by a process known as chemotaxis. At the site of inflammation, tissue damage and complement activation cause the release of chemotactic peptides such as C5a. Complement activation products are also responsible for causing degranulation of phagocytic cells, mast cells and basophils, smooth muscle contraction and increases in vascular permeability. The traversing of leukocytes from the bloodstream to extravascular sites of inflammation or immune reaction involves a complex but coordinated series of events. At the extravascular site of infection or tissue injury, signals are generated such as bacterial endotoxins, activated complement fragments or proinflammatory cytokines such as interleukin 1 (DL-1), interleukin 6 (IL-6), and tumor necrosis factor (TNF) which activate leukocytes and/or endothelial cells and cause one or both of these cell types to become adhesive. Initially, cells become transiently adhesive (manifested by rolling) and later, such cells become firmly adhesive (manifested by sticking). Adherent leukocytes travel across the endothelial cell surface, diapedese between endothelial cells and migrate through the subendothelial matrix to the site of inflammation or immune reaction. Although leukocyte traversal of vessel walls to extravascular tissue is necessary for host defense against foreign antigens and organisms, leukocyte-endothelial interactions often have deleterious consequences for the host. For example, during the process of adherence and transendothelial migration, leukocytes release oxidants, proteases and cytokines that directly damage endothelium or cause endothelial dysfunction. Once at the extravascular site, emigrated leukocytes further contribute to tissue damage by releasing a variety of inflammatory mediators. Moreover, single leukocytes sticking within the capillary lumen or aggregation of leukocytes within larger vessels are responsible for microvascular occlusion and ischemia. Leukocyte-mediated vascular and tissue injury has been implicated in pathogenesis of a wide variety of clinical disorders such as acute and chronic allograft rejection, vasculitis, rheumatoid and other forms of inflammatory based arthritis, inflammatory skin diseases, adult respiratory distress syndrome, ischemia-reperfusion syndromes such as myocardial infarction, shock, stroke, organ transplantation, crush injury and limb replantation.

Many other serious clinical conditions involve underlying inflammatory processes in humans. For example, multiple sclerosis (MS) is an inflammatory disease of the central nervous system. In MS, circulating leukocytes infiltrate inflamed brain endothelium and damage myelin, with resultant impaired nerve conduction and paralysis. In the case of concussions, these head injuries cause accumulated damage that triggers inflammation.

Various anti-inflammatory drugs are currently available for use in treating conditions involving underlying inflammatory processes. Their effectiveness however, is widely variable and there remains a significant clinical unmet need. This is especially true in the aforementioned diseases where available therapy is either of limited effectiveness or is accompanied by unwanted side effect profiles. The present disclosure provides solutions to these problems.

### BRIEF SUMMARY

Disclosed are means, methods and compositions of matter useful for stimulation of regenerative and/or anti-inflammatory activity in cells, tissues, and/or organs of a recipient by administration of fibroblasts and minocycline and/or analogues thereof. In one embodiment, fibroblasts are administered to treat or prevent a neurological disorder, wherein said fibroblasts are co-administered with minocycline and/or analogues thereof, including at least in some cases to an amount in the recipient effective to decrease inflammatory activity and enhancing the ability to induce production of one or more regenerative cytokines. In some embodiments, the combination of minocycline (and/or analogues thereof) and fibroblasts is administered subsequent to a central nervous system injury that is acute, such as stroke. In other embodiments, minocycline and/or analogues thereof are administered together with fibroblasts for treatment of chronic injuries, such as chronic traumatic encephalopathy (CTE), for example.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 shows synergy of fibroblasts and minocycline at suppressing inflammation as measured by IL-1beta. The bars from left to right are control, fibroblasts, minocycline, and a combination of fibroblasts and minocycline.

FIG. 2 shows synergy of fibroblasts and minocycline at suppressing inflammation as measured by TNFalpha. The bars from left to right are control, fibroblasts, minocycline, and a combination of fibroblasts and minocycline.

FIG. 3 shows synergy of fibroblasts and minocycline at suppressing inflammation as measured by IL-6. The bars from left to right are control, fibroblasts, minocycline, and a combination of fibroblasts and minocycline.

FIG. 4 demonstrates that fibroblasts augment the ability of minocycline to induce T regulatory cells.

### DETAILED DESCRIPTION

### I. Definitions

Unless defined otherwise, all technical and scientific terms used herein have the same meaning as commonly understood by those of ordinary skill in the art to which this disclosure belongs. Although any methods and materials similar or equivalent to those described herein can be used in the practice or testing of the present disclosure, the preferred methods and materials are described. Generally, nomenclatures utilized in connection with, and techniques of, cell and molecular biology and chemistry are those well-known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

The term “neuroprotective” means a treatment that has an effect that reduces, arrests, or ameliorates nervous insult and is protective, resuscitative or revivative for nervous tissue that has suffered nervous insult, such as in the case of a suspected neurodegenerative disease. It may include reduction of neuronal death or loss of function in diseases such as Alzheimer's Disease (AD), age-associated memory impairment, mild cognitive impairment, cerebrovascular dementia, etc. The present term is associated with neurodegenerative diseases, which may be diagnosed by known methods, including biomarkers, PET imaging, etc. For examples of determining the existence and progression of these neurodegenerative diseases, see: Mueller et al., “Evaluation of treatment effects in Alzheimer's and other neurodegenerative diseases by MRI and MRS,” NMR Biomed. 2006 October; 19(6): 655-668.

A “pharmaceutically acceptable” excipient is one that is suitable for use with humans and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

In some embodiments “therapeutically effective amount” refers to a safe and effective amount of a component effective to yield the desired therapeutic response, for example, an amount effective to prevent or treat (ameliorate) neurodegeneration, memory loss, and/or dementia.

“Allogeneic,” as used herein, refers to cells of the same species that differ genetically from cells of a host.

“Autologous,” as used herein, refers to cells derived from the same subject. The term “engraft” as used herein refers to the process of stem cell incorporation into a tissue of interest in vivo through contact with existing cells of the tissue.

“Approximately” or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Carrier” or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, the therapeutic agent is administered continuously over a predetermined period. In some embodiments, the therapeutic agent is administered once a day (QD) or twice a day (BID).

The term “culture-expanded population” means a population of cells whose numbers have been increased by cell division in vitro. This term may apply to stem cell populations and non-stem cell populations alike, including fibroblasts.

The term “passaging” refers to the process of transferring a portion of cells from one culture vessel into a new culture vessel.

The term “cryopreserve” refers to preserving cells for long term storage in a cryoprotectant at low temperature.

The term “master cell bank” refers to a collection of cryopreserved cells. Such a cell bank may comprise fibroblasts, stem cells, non-stem cells, and/or a mixture of stem cells and non-stem cells. Any cells may be obtained from and/or deposited in a master cell bank.

The disclosure encompasses means of “programming” the immune system to suppress autoimmunity through stimulation of T regulatory cells using minocycline as a means of activating T regulatory cells, and/or inducing proliferation, and/or inducing their de novo generation.

### II. Embodiments

The disclosure encompasses the treatment and prevention of medical conditions associated with the brain, including from injury and/or disease and for any mammal, including humans, dogs, cats, horses, and so forth. Methods of the disclosure treat or prevent neurological damage. The medical condition may be a neurological disorder. Any kind of brain injury may be treated or prevented, including traumatic brain injury. The injury may comprise hematoma, hemorrhage, concussion, edema, a mixture thereof, and so forth. Types of traumatic brain injuries include brain contusion, second impact syndrome, Coup-Contrecoup brain injury, shaken baby syndrome, and/or penetrating injury.

The medical conditions may be the result of a single injury or repeated injury, in some cases. The injuries may be from physical contact, including as the result of a vocation and/or sport. The medical condition may be a neurological disorder. Any injuries may have occurred at any time during the life of the individual, including years, months, days, or weeks prior to the onset of any symptom. In particular embodiments, the medical condition is chronic traumatic encephalopathy (CTE), including dementia pugilistica. The individual may be an athlete, including one that is involved recreationally or professionally in football, boxing, wrestling, soccer, hockey, lacrosse, basketball, and so forth. The individual may have a medical condition such as a head injury related to their job, such as a construction worker, first responder, warehouse worker, and so forth.

An individual may be provided the therapy of the disclosure prior to exposure to an environment subject to a risk for head trauma, such as a sporting field of play, a construction site, etc. Any administration of the therapy, whether prior to a head injury and/or following a head injury, may be a single or multiple administrations. Any duration of time between administrations may be utilized, including on the order of hours, days, weeks, months, or years. In specific embodiments, the therapy is provided to the individual within 1-60 minutes of the injury, within 1-24 hours of the injury, within 1-4 weeks of the injury, within 1-12 months of the injury, or within 2 or more years following the injury.

In some cases, fibroblasts are exposed to one or more anti-inflammatory agents prior to use, in order to augment therapeutic activity of the fibroblasts. In specific embodiments, there is co-administration of one or more anti-inflammatory agents, such as NF-kappa B inhibitors, together with fibroblasts in order to augment therapeutic activity of the fibroblasts. In one embodiment, the fibroblasts administered are allogeneic fibroblasts and the anti-inflammatory agent that is co-administered is minocycline and/or analogues thereof (such as Tigecycline). Any compositions encompassed herein may be provided to an individual at risk of a head injury, such as prior to playing a sport or entering a hazardous job environment.

In one aspect, the disclosure addresses differentiation of naive T cells into stable regulatory T-cells (Tregs) using administration of fibroblasts and minocycline (and/or analogues thereof), and/or combinations of fibroblasts with one or more other immune regulatory agents, such as low dose interleukin-2. This disclosure is based, in part, on the observation that administration of minocycline (and/or analogues thereof) possesses ability to modulate T regulatory cell numbers in healthy animals and in animals suffering from neurological issues such as chronic traumatic encephalopathy. Accordingly, the inventors investigated the use of minocycline to stimulate T regulatory cells, as well as induce augmented immune suppressive activity of T regulatory cells. The results, described in more detail below, disrupt general paradigms that minocycline acts as a direct anti-inflammatory agent, but instead induces a state of “active immune tolerance”. Indeed it is reasonable to believe that the current discovery is markedly different than previous findings based on the fact that T regulatory cells can induce a state of “infectious tolerance” in which the tolerogenic process maintains itself after cessation of administration of the therapeutic agent. Such maintenance of a tolerogenic state has been previously described by numerous investigators [1-4]. The utilization of minocycline newly discovered property of stimulating T regulatory cells is applicable to a wide variety of diseases. As the utility of this approach has become clear, one likely advantage to such therapeutic compounds is the enhanced likelihood for tolerance (e.g., reduced toxicity and side-effects) in the human (or other) subject. As used herein, the term “naive T cells” refers to lymphocytes that are typically derived from the thymus and express T cell receptors. The naive T cells have typically undergone the basic development in the bone marrow and further undergone the positive and negative processes of selection in the thymus. However, naive T cells have not encountered their cognate antigens yet in the periphery. The terms “activating” and/or “differentiation” refers to the process in which the naive T cell are caused to further develop into one of at least four distinct lineages of T cells characterized by distinct expression profiles and functions in vivo. The term “activated” and/or “differentiated” can refer to the cell that had previously been naive but now has had an induction of specific gene expression such that it is identifiable as a particular activated/differentiated lineage. The terms can also refer to cells produced from the expansion of a T cell into a multitude of progeny cells by cell-division and which retain the identifiable markers for the particular activated/differentiated lineage. Thus, “activating a naive T cell” can refer to the production of an expanded cell population of differentiated T cells from the initial naive T cell, as well as the initial T cell after gene transcription has been induced.

For any embodiment, it can be readily determined the minimal amount of minocycline (and/or analogues thereof) required to effect activation of the naive T cell into the desired differentiated T cell.

In one embodiment, minocycline (and/or analogues thereof) is used to treat the naive T cell that is differentiated into a cell with increased expression of FoxP3 compared to the naive T cell. The term “FoxP3” refers to a transcription factor also referred to as “forkhead box P3” or “scurfin”. FoxP3 protein belongs to the forkhead/winged-helix family of transcriptional regulators. In regulatory T cell model systems, FoxP3 occupies the promoters for genes involved in regulatory T-cell function, and may repress transcription of key genes following stimulation of T cell receptors. Accordingly, FoxP3 is known as a master regulator in the development of regulatory T cells (Tregs), which are involved in tolerance of antigens in the periphery and generally promote a protection against an inflammatory response. Examples of the FoxP3 protein include human (Entrez #: 50943; RefSeq (mRNA): NM_001114377; RefSeq (amino acid): NP_001107849) and mouse (Entrez #: 20371; RefSeq (mRNA): NM_001199347; RefSeq (amino acid): NP_001186276). Many other FoxP3 protein and gene homologs are known for vertebrate animals, and their expression can be readily determined. As used herein, the term “increased” refers to a level of expression of the FoxP3 transcription factor that is detectably greater than that in a naive T cell, such as the initial naive T cell that is being differentiated, or other naive T cell obtained from the same individual (or an individual of the same species) as that as the initial naive T cell. Increased expression can be determined in terms of transcription of the underlying foxp3 gene or levels of functional FoxP3, using routine and established methods known in the art.

In one embodiment, the naive T cell is differentiated into a T regulatory cell (Treg). The term “Treg” refers to a lineage of T cells that promote or maintain tolerance to antigens, typically to self-antigens. Tregs have been previously referred to as “suppressor T cells.” Tregs generally suppress or downregulate induction and proliferation of effector T cells. As indicated above, Treg cells are typically characterized by the positive or increased expression of FoxP3. Tregs are also characterized by the additional positive or increased expression of CD4 and CD25. Thus, in one embodiment, the Treg is characterized by a state of CD4+, CD25+ and FoxP3+ expression.

In other embodiments, the contacting of the naive T cell results in an inhibition of a “Th17” inflammation phenotype by the differentiated T cells. For instance, the contacting of the naive T cell results in an inhibition or decrease in the expression of ROR.gamma.T, which is a marker for the Th17 (pro-inflammatory) phenotype of activated T cell normally involved in mucosal immunity In one embodiment herein, the naive T cell can be contacted in vitro in a culture medium. Typically, the culture medium contains factors commonly known to support and maintain T cell viability. The medium can also contain additional ingredients that are also known to promote T cell activation toward the desired differentiated lineage. Such additional ingredients are often referred to as “skewing” ingredients. Skewing factors can also include other microbiota metabolites (such as short-chain fatty acids, bile acids, polysaccharide A), dietary derived compounds (such as n3 polyunsaturated fatty acids, retinoic acid, and other vitamin-derivatives (VitD, VitC, etc.), polyphenols, quercetin, resveratrol, NSAIDS, TGF-.beta., IL-10, rapamycin, and IL-2. Other skewing factors that are useful for this purpose include curcumin, metformin/AMPK activators, PI3-kinase/Akt inhibitors, and PPAR agonists, as are known in the art. The invention teaches that minocycline augments ability of “skewing factors” to generate enhanced numbers of T regulatory cells.

In another aspect, the present disclosure provides a method of producing a Treg cell. In one embodiment, the method comprises contacting a naive T cell in vitro with a minocycline, wherein said minocycline can be contacted with the naive T cell as a component (e.g., additive) of a standard culture medium, as described above. The method can comprise the further culture and/or expansion of the activated T cell in its differentiated Treg state.

As described below, the inventors have demonstrated that the Tregs that are induced in vitro (“iTregs”) using the disclosed minocycline possess new features over induced Tregs (“iTregs”) produced using existing techniques. For example, the inventors have demonstrated that the iTregs resulting from the application of the TDMMs, such as indole, resulted in a stable iTreg that did not revert to a Th17 phenotype even in a “pro-inflammation” environment. Thus, in another aspect, the disclosure provides an induced T regulatory cell (iTreg). The iTreg is produced by the methods described herein. In some embodiments, the iTreg is produced by contacting a naive T cell with minocycline. The iTreg can be the initial T cell after activation has occurred or a progeny cell in the differentiated state after expansion has occurred through one or more rounds of cell division from the initial T cell. In some embodiments, the iTreg exhibits increased stability in the Treg lineage as compared to iTregs that are induced using conventional means. For example, IL-4, IL-6, and IL-23 are all known to reduce typical Treg stability. This obstacle is overcome by iTregs. Accordingly, the iTreg lineage is less susceptible to induced instability by IL-4, IL-6, and IL-23. In some embodiments, the iTregs are distinguished from typical Tregs by a relative increased expression of CTLA4, CD62L, CD25, higher Foxp3, alpha4beta7, and/or CCR9, which can readily be determined by routine testing.

In another aspect, the present disclosure provides a method of increasing the stability of Treg cells by administration of minocycline (and/or analogues thereof). In specific embodiments, this refers to the lowered susceptibility of the Tregs to alter the Treg specific expression profiles in the context of pro-inflammatory cytokines and signaling, such as IL-4, IL-6, and IL-23, and the like. The Treg cells can be induced Tregs (iTregs) such as produced by the novel methods described herein or by existing methods in the art. Alternatively, the Tregs can be naturally occurring Tregs (nTregs). The term “nTregs” refers to the Tregs existing in vivo without prior in vitro intervention or transfer and are typically obtained from the thymus in humans. This method can be carried out in vitro by isolating and the Treg population, or alternatively expanding an iTreg population already ex vivo, and exposing the Tregs to the minocycline, or a precursor, prodrug, analogue, or acceptable salt thereof, as described herein. In some cases, if the target population is an iTreg population produced by the novel methods described herein, the iTregs will have already been exposed to the minocycline, or a precursor, prodrug, or acceptable salt thereof, and may or may not have additional exposure.

In another aspect, the present disclosure provides a method of reducing, preventing, ameliorating, attenuating, and/or otherwise treating inflammation in a subject in need thereof. General methods of using isolated or ex vivo/in vitro-differentiated Treg cells as part of adoptive T cell therapy to address inflammatory-related diseases are known. The method of the present aspect comprises administering to the subject the iTreg described immediately above, i.e., which is produced by contacting a naive T cell with minocycline (and/or analogues thereof). In some embodiments, the subject suffers from or is susceptible to excessive or deleterious inflammation. In some embodiments, the subject has or is susceptible to allergies, inflammatory bowel disease, colitis, NSAID-enteropathy/ulceration, psoriasis, rheumatism, graft-versus-host disease, lupus, multiple sclerosis, and the like. In some embodiments, the subject has or is susceptible to a disease characterized by the role of mTor, stat3, akt, erk, jnk, stat5, and/or smad2/3, which are targets of indole. Additionally or alternatively, the subject may suffer from deleterious inflammation due to a cancer or infection from a microbial or parasitic pathogen. The iTreg can be formulated for administration through any appropriate route according to known standards and methods. For example, the iTregs can be formulated for intra-peritoneal (IP), intravenous (IV), topical, parenteral, intradermal, transdermal, oral (e.g., via liquid or pill), inhaled (e.g., intranasal mist), and other appropriate routes of administration. In some embodiments, administration is directly to a mucosal region of the subject, such as in the digestive tract.

In some embodiments, the method comprises inducing the development of Tregs in vivo as described herein. In such embodiments, the subject can be administered an effective amount of minocycline, or a precursor, prodrug, analogue, or acceptable salt thereof. Administration of the minocycline (and/or analogue thereof) can be in any appropriate route of administration. For example, the minocycline and/or analogue(s) can be administered by intra-peritoneal (IP), intravenous (IV), topical, parenteral, intradermal, transdermal, oral (e.g., via liquid or pill), rectal, or respiratory (e.g., intranasal mist) routes. In preferred embodiments, the minocycline is ingested, e.g., via liquid or pill, etc. to facilitate delivery of the minocycline to the intestinal tract.

In some embodiments of the disclosure, the drug, such as minocycline or a prodrug of minocycline or analogue of minicycline, is delivered systemically to achieve therapeutically effective plasma concentrations in a patient. However, drug oral dosage forms, including those comprising minocycline, must overcome several obstacles in order to achieve a therapeutically-effective systemic concentration. First, tetracyclines are generally highly lipophilic. Their limited water solubility thereby restricts the amount of tetracycline available for absorption in the gastrointestinal tract. Second, minocycline, as with the other tetracyclines, undergoes substantial first-pass metabolism when absorbed from the human gastrointestinal tract. Finally, the oral bioavailability of any product is further diminished when a patient suffers from nausea or emesis, as either the patient avoids taking his oral medications or the oral dosage form does not remain in the gastrointestinal tract for a sufficient period of time to release the entire dose and achieve a therapeutic concentration. Therefore, in view of the foregoing, it would be desirable to systemically deliver therapeutically effective amounts of a tetracycline, such as minocycline or minocycline prodrug, to a mammal in need thereof for the treatment of one or more medical conditions responsive to tetracycline, including pancreatic cancer, pancreatitis, pain, nausea or appetite stimulation, by a route of administration that does not depend upon absorption from the gastrointestinal tract of the mammal. One non-oral route of administration for the systemic delivery of minocycline is transdermal administration. In addition, the epidermis and dermis of many mammals, such as humans and guinea pigs, contains enzymes which are capable of metabolizing active pharmaceutical agents which pass through the stratum corneum. The metabolic process occurring in the skin of mammals, such as humans, can be utilized to deliver pharmaceutically effective quantities of a tetracycline, such as minocycline, to the systemic circulation of a mammal in need thereof. Described herein are prodrugs of tetracycline, such as minocycline prodrugs, and compositions comprising prodrugs of tetracycline that can be transdermally administered to a mammal, such as a human, so that the metabolic product resulting from metabolism in the skin is the tetracycline which is systemically available for the treatment of a medical condition responsive to tetracycline, for example pancreatic diseases, such as pancreatitis and pancreatic cancer. Unfortunately, due to its highly lipophilic nature, minocycline is poorly absorbed through membranes such as the skin of mammals, including humans. Therefore, the success of transdermally administering therapeutically effective quantities of minocycline to a mammal in need thereof within a reasonable time frame and over a suitable surface area has been substantially limited.

The use of minocycline for suppression of inflammation has previously been described in the art. The invention provides means of utilizing the anti-inflammatory effects of minocycline for stimulation of Treg cells. Once Treg cells are generated, the invention teaches that such Treg cells may be expanded.

The disclosure provides means of utilizing minocycline to prevent unwanted immune responses. For example, in pregnancy, “tolerogenic antigen presentation” occurs only through the indirect pathway of antigen presentation [5]. Other pathways of selective tolerogenesis in pregnancy include the stimulation of Treg cells, which have been demonstrated essential for successful pregnancy [6]. The disclosure, in one embodiment, teaches the modification of fibroblasts by transfection with MHC or MHC—like molecules in order to create an antigen presenting cell from said fibroblasts, wherein the antigen presenting cell is capable of inducing antigen-specific tolerance when administered into a host at a therapeutically sufficient concentration and frequency. In the context of cancer, depletion of tumor specific T cells, while sparing of T cells with specificities to other antigens has been demonstrated by the tumor itself or tumor associated cells [7-10]. This is the mechanism why which cancer can selectively induce a “hole in the repertoire” while allowing the host to be generally immunocompetent. Additionally, Treg cells have been demonstrated to actively suppress anti-tumor T cells, perhaps as a “back up” mechanism of tumor immune evasion [11-13]. At a clinical level the ability of tumors to inhibit peripheral T cell activity has been associated in numerous studies with poor prognosis [14-16]. Accordingly, in one embodiment of the disclosure, the utilization of molecules that stimulate generation of Treg, as well as administration of molecules that expand Tregs which have been generated, are utilized. In one embodiment, fibroblasts are transfected with one or more autoantigens together with interleukin-2 in order to enhance Treg generation. In other embodiments, interleukin 2 is administered systemically in order to enhance in vivo proliferation of Tregs.

In one embodiment, tolerance is induced to autoantigens that are part of CTE initiation and progression. It is believed by the inventors that CTE possesses an autoimmune component and through suppression of this one can accelerate efficacy of fibroblast therapy. Natural example of tolerance that is utilized by the disclosure as a template for us of minocycline induced T regulatory cells is oral tolerance. Oral tolerance is the process by which ingested antigens induce generation of antigen-specific TGF-beta producing cells (called “Th3” by some) [17-19], as well as Treg cells [20, 21]. Ingestion of antigen, including the autoantigen collagen II [22], has been shown to induce inhibition of both T and B cell responses in a specific manner [23, 24]. It appears that induction of regulatory cells, as well as deletion/anergy of effector cells is associated with antigen presentation in a tolerogenic manner [25]. Remission of disease in animal models of RA [26], multiple sclerosis [27], and type I diabetes [28], has been reported by oral administration of autoantigens. Furthermore, clinical trials have shown signals of efficacy of oral tolerance in autoimmune diseases such as rheumatoid arthritis [29], autoimmune uveitis [30], and multiple sclerosis [31]. In all of these natural conditions of tolerance, common molecules and mechanisms seem to be operating. Accordingly, a natural means of inducing tolerance would be the administration of a “universal donor” cell with tolerogenic potential that generate molecules similar to those found in physiological conditions of tolerance induction. In some embodiments, oral tolerance is utilized together with the autoantigen transfected fibroblasts of the invention. For example, if a patient with type 1 diabetes is treated, the patient is administered minocycline, as well as cells that have been transfected with a diabetes specific autoantigen such as GAD65, additionally said cells may be transfected with tolerogenic molecules such as IL-10, and when said cell are administered, orally delivered GAD65 may be utilized in order to enhance the tolerogenic processes. In another embodiment, the invention teaches the transfection of cell with autoantigens combined with molecules associated with oral tolerogenesis such as TGF-beta.

The disclosure encompasses the previously unexpected finding that administration of minocycline and other compounds associated with inhibition of inflammation are able to potently augment regenerative activities of fibroblast cells. In some embodiments the dose of minocycline is adjusted based on the need of the individual, conditions of the individual and the underlying disease. Various doses may be used including comprises between about 10-100 mg (or between about 1 mg and 400 mg, between about 10 mg and 300 mg, between about 10 mg and 150 mg, between about 10 mg and 120 mg, between about 10 mg and 100 mg, between about 20 mg and 400 mg, between about 20 mg and 300 mg, between about 20 mg and 200 mg, between about 30 mg and 400 mg, between about 30 mg and 300 mg, between about 30 mg and 200 mg, between about 30 mg and 100 mg, between about 50 mg and 400 mg, between about 50 mg and 300 mg, between about 50 mg and 200 mg, between about 50 mg and 100 mg, between about 10 mg and 90 mg, between about 10 mg and 80 mg, between about 10 mg and 70 mg, between about 10 mg and 60 mg, between about 10 mg and 50 mg, etc.) of minocycline and about 10-400 mg of minocycline (e.g., between about 50-200 mg, between about 10-300 mg, between about 10-200 mg, between about 10-150 mg, between about 10-100 mg, between about 10-90 mg, between about 10-80 mg, between about 10-70, between about 10-60, between about 10-50 mg, between about 20-400 mg, between about 20-300, between about 20-200 mg, between about 20-100 mg, between about 20-90 mg, between about 30-500 mg, between about 30-400 mg, between about 30-300 mg, between about 30-200 mg, between about 30-100 mg, etc.; between about 40-500 mg, between about 40-400 mg, between about 40-300 mg, between about 40-200 mg, between about 40-100 mg, between about 50-500 mg, between about 50-400 mg, between about 50-300 mg, between about 50-100 mg, between about 50-80 mg, etc.).

In one embodiment of the disclosure, minocycline is used based on properties known in the art to possess therapeutically relevant activities. For example, it has been shown that minocycline is capable of inhibiting microglial activation. In one example, it was shown that this antibiotic protects hippocampal neurons against global ischemia in gerbils. Minocycline increased the survival of CA1 pyramidal neurons from 10.5% to 77% when the treatment was started 12 h before ischemia and to 71% when the treatment was started 30 min after ischemia. The survival with corresponding pre- and posttreatment with doxycycline was 57% and 47%, respectively. Minocycline prevented completely the ischemia-induced activation of microglia and the appearance of NADPH-diaphorase reactive cells, but did not affect induction of glial acidic fibrillary protein, a marker of astrogliosis. Minocycline treatment for 4 days resulted in a 70% reduction in mRNA induction of interleukin-1beta-converting enzyme, a caspase that is induced in microglia after ischemia. Likewise, expression of inducible nitric oxide synthase mRNA was attenuated by 30% in minocycline-treated animals [32]. The benefits of minocycline from protection against inflammation associated neural cell death where seen in another study in which Minocycline (0.02 microm) significantly increased neuronal survival in mixed spinal cord (SC) cultures treated with 500 microm glutamate or 100 microm kainate for 24 hr. Treatment with these excitotoxins induced a dose-dependent proliferation of microglia that was associated with increased release of interleukin-1beta (IL-1beta) and was followed by increased lactate dehydrogenase (LDH) release. The excitotoxicity was enhanced when microglial cells were cultured on top of SC cultures. Minocycline prevented excitotoxin-induced microglial proliferation and the increased release of nitric oxide (NO) metabolites and IL-1beta. Excitotoxins induced microglial proliferation and increased the release of NO metabolites and IL-1beta also in pure microglia cultures, and these responses were inhibited by minocycline. In both SC and pure microglia cultures, excitotoxins activated p38 mitogen-activated protein kinase (p38 MAPK) exclusively in microglia. Minocycline inhibited p38 MAPK activation in SC cultures, and treatment with SB203580, a p38 MAPK inhibitor, but not with PD98059, a p44/42 MAPK inhibitor, increased neuronal survival. In pure microglia cultures, glutamate induced transient activation of p38 MAPK, and this was inhibited by minocycline [33]. One of the interesting traits of minocycline is that it can be used at low concentrations. In one study, it was shown that nanomolar concentrations of minocycline protect neurons in mixed spinal cord cultures against NMDA excitotoxicity. NMDA treatment alone induced microglial proliferation, which preceded neuronal death, and administration of extra microglial cells on top of these cultures enhanced the NMDA neurotoxicity. Minocycline inhibited all these responses to NMDA. Minocycline also prevented the NMDA-induced proliferation of microglial cells and the increased release of IL-1beta and nitric oxide in pure microglia cultures. Finally, minocycline inhibited the NMDA-induced activation of p38 mitogen-activated protein kinase (MAPK) in microglial cells, and a specific p38 MAPK inhibitor, but not a p44/42 MAPK inhibitor, reduced the NMDA toxicity [32].

In one embodiment of the disclosure, minocycline is utilized to suppress microglial activation prior to, concurrent with, or subsequent to administration of fibroblasts in order to allow for said fibroblasts to induce a therapeutic effect on the brain in absence of the chronic inflammation induced by activated microglia [34-45]. This is useful in conditions such as CTE in which microglial activation has previously been demonstrated to be found, and also to be associated with various pathologies of CTE, such as depression.

In some embodiments, minocycline is administered in order to modify interactions between T cells and microglia in the context of a patient receiving fibroblasts [46-57]. Modulation of T cell activity may be desired to enhance survival of allogeneic fibroblasts [58]. Alternatively, the modulation of T cell activity may be utilized in order for said T cells to produce trophic factors that enhance activity of fibroblasts.

The study of neurological pathologies such as CTE teaches that many preclinical observations strongly suggest that neuroprotective approaches may also confer beneficial effects. Since many lines of evidence suggest that several pathological pathways leading to a cell death are activated in neurological disorders, simultaneously targeting different pathways should be a rational approach to treatment. It is provided herein evidence fibroblast activity may be augmented using, in one embodiment of the disclosure, a three-drug cocktail consisting of minocyline, an antimicrobial agent with antiapoptotic and anti-inflammatory properties that blocks microglial activation, riluzole, a glutamate antagonist and nimodipine, a voltage gated calcium channel blocker, exerted remarkable neuroprotection in a mouse model of amyotrophic lateral sclerosis.

For use within the current disclosure, minocycline is a semisynthetic tetracycline derivative that effectively crosses blood-brain barrier and it is extensively used in human with relatively little side effects. It has been suggested that minocycline exerts neuroprotective effects by preventing microglial activation, reducing the induction of caspase-1 thereby decreasing the level of mature proinflammatory cytokine IL-1.beta. and inhibiting cytochrome-c release from mitochondria [59-68]. In addition, it has been shown that minocycline, doxycycline and their non-antibiotic derivatives (chemically modified tetracyclines) inhibit matrix metalloproteases, nitric oxide synthases, protein tyrosine nitration, cyclooxygenase-2 and prostaglandine E2 production. Recent studies performed with primary neurons and purified microglial cultures demonstrated that minocycline may also confer neuroprotection through inhibition of excitotoxin-induced microglial activation. Minocycline inhibits glutamate- and kainate-induced activation of p38 MAPK, exclusively activated in microglia. In some embodiments of the invention, minocycline is administered together with an anti-glutaminergic drug such as Riluzole, a glutamate antagonist, is the only drug currently approved for therapy of ALS with only marginal effects on survival (Rowland, L. P. & Shneider, N. A. (2001) N. Eng. J. Med. 344, 1688-1699). In two controlled clinical trials it increased survival of ALS patients by 3-6 months. Although the precise mechanism of action of riluzole has not been fully elucidated, it appears to involve interference with excitatory amino acid (EAA) in the CNS, possibly through inhibition of glutamic acid release, blockade or inactivation of sodium channels and/or activation of G-protein coupled transduction pathways. When tested as a single therapy in SOD1 mutant mice it increased survival for 13-15 days without affecting the onset of disease (Gurney, M. E., et al. (1996) Ann. Neurol. 39,147-157).

In some embodiments of the disclosure, minocycline is utilized to generate tolerogenic dendritic cells in vivo [69], wherein the tolerogenic dendritic cells are utilized to induce T regulatory cells, wherein said T regulatory cells suppress inflammation and allow for enhanced activity of transplanted fibroblasts.

For administration of minocycline, or derivatives thereof, in some embodiments when the agent of the present disclosure, or the concomitant drug of the agent of the present disclosure with another agent, is used for the above-described purpose, it is generally administered systemically or topically in the oral or parenteral form.

Its dose varies depending on the age, body weight, symptoms, therapeutic effect, administration method, treating period and the like, but is usually within the range of from 1 ng to 100 mg per adult per once, from once to several times a day by oral administration, or within the range of from 0.1 ng to 50 mg per adult per once, from once to several times a day, from once to several times a week, or from once to several times in 3 months by parenteral administration in the form of a persistent preparation, or continuously administered into a vein within the range of from 1 hour to 24 hours a day. Since the dose varies under various conditions as a matter of course as described above, there is a case in which a smaller dose than the above range is sufficient or a case which requires the administration exceeding the range. When the agent of the present invention, or the concomitant drug of the agent of the present invention with other agent, is administered, it is used as solid preparations for internal use or liquid preparations for internal use for oral administration, or as injections, subcutaneous or intramuscular injections, external preparations, suppositories, eye drops, inhalations, medical device-containing preparations and the like for parental administration. The solid preparation for internal use for use in the oral administration includes tablets, pills, capsules, powders, granules and the like. Hard capsules and soft capsules are included in the capsules. In such a solid preparation for internal use, one or more active substances are used as such, or mixed with a filler (lactose, mannitol, glucose, microcrystalline cellulose, starch, etc.), a binder (hydroxypropylcellulose, polyvinyl pyrrolidone, magnesium aluminometasilicate, etc.), a disintegerating agent (calcium cellulose glycolate, etc.), a lubricant (magnesium stearate, etc.), a stabilizing agent, a solubilization assisting agent (glutamic acid, aspartic acid, etc.) and the like and used by making the mixture into a pharmaceutical preparation. If necessary, this may be coated with a coating agent (sucrose, gelatin, hydroxypropylcellulose, hydroxypropylmethylcellulose phthalate, etc.), or coated with two or more layers. Further capsules of an absorbable substance such as gelatin are included.

The liquid preparation for internal use for use in the oral administration includes pharmaceutically acceptable solutions, suspensions, emulsions, syrups, elixirs and the like. In such a liquid preparation, one or more active ingredient are dissolved, suspended or emulsified in a generally used diluent (purified water, ethanol, a mixed solution thereof, etc.). In addition, this liquid preparation may contain a moistening agent, a suspending agent, an emulsifying agent, a sweetener, a flavor, an aromatic, a preservative, a buffer and the like. Dosage forms for external use for use in parenteral administration include, for example, ointments, gels, creams, fomentations, adhesive preparations, liniments, sprays, inhalations, sprays, aerosols, eye drops, nasal drops and the like. In addition, these may be sealed with a biodegradable polymer and used as medical devices (surgical suture, a bolt for use in bone fracture treatment, etc.). They contain one or more active ingredient and are prepared by a conventionally known method or based on a generally used formula. In addition to the generally used diluents, the sprays and inhalations may contain stabilizers such as sodium hydrogen sulfite and buffer agents capable of giving tonicity, for example, tonicity agents such as sodium chloride, sodium citrate and citric acid. Production methods of sprays are illustratively described in, for example, U.S. Pat. Nos. 2,868,691 and 3,095,355. Solutions, suspensions, emulsions and solid injections which are used by dissolving or suspending in a solvent prior to use are included in the injections for parenteral administration. The injections are used by dissolving, suspending or emulsifying one or more active ingredients in a solvent. These injections may be injected into a vein, an artery, muscle, under the skin, into the brain, a joint, a bone and other topical regions of organs, or directly administered using a needle-equipped blood vessel catheter or the like. As the solvent, for example, distilled water for injection, physiological saline, plant oil, alcohols such as propylene glycol, polyethylene glycol and ethanol, and combinations thereof are used. In addition, such injections may contain a stabilizer, a solubilization assisting agent (glutamic acid, aspartic acid, Polysorbate 80 (registered trade mark), etc.), a suspending agent, an emulsifying agent, a soothing agent, a buffer agent, a preservative and the like. These are produced by sterilizing at the final step or by an aseptic operation. In addition, it is possible to prepare an aseptic solid preparation, such as a freeze-dried preparation, and use it by dissolving in sterilized or aseptic distilled water for injection or other solvent prior to its use.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synergy of Fibroblasts and Minocycline at Suppressing Inflammation

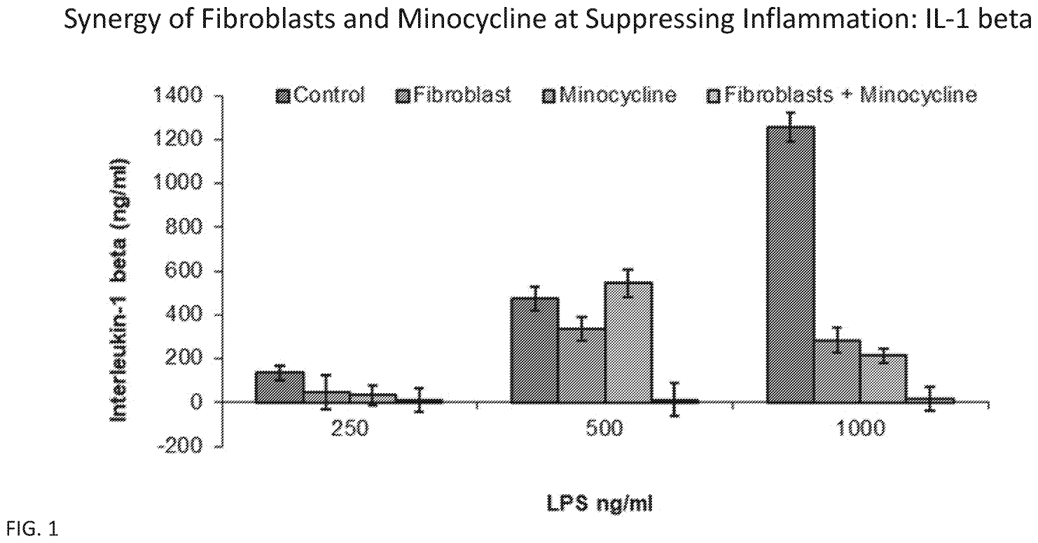

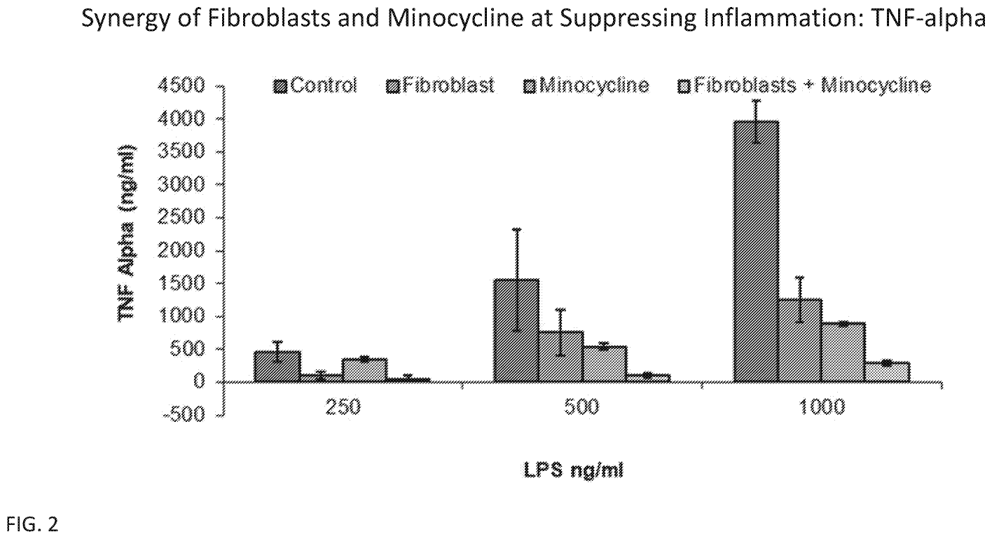

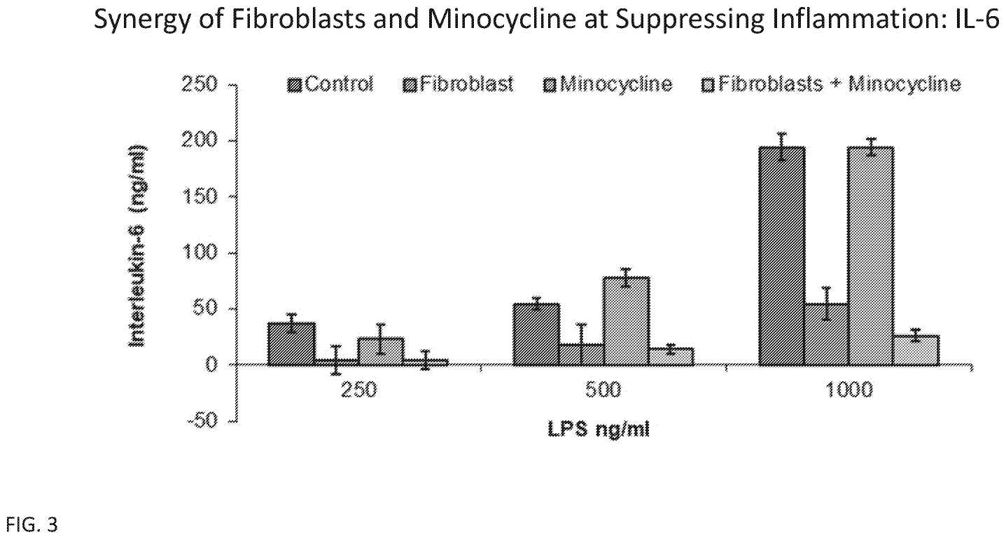

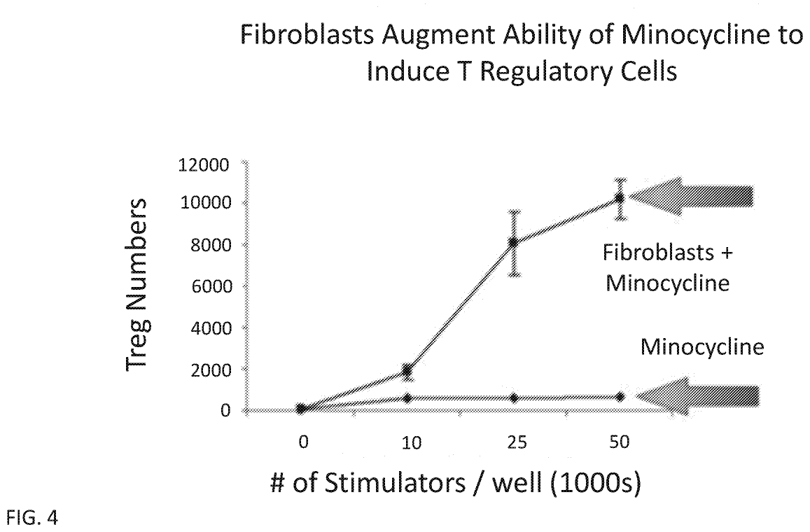

Three concentrations of lipopolysaccharide (that activates TLR4, a receptor associated with CTE) were utilized to mimic TLR4 activation and were added to 96-well plates that had adherent monocytes at confluence. The following were added: Fibroblasts alone, Minocycline alone, and Fibroblasts+minocycline. Assessment of inflammatory cytokines was examined: IL-1 beta (FIG. 1), TNF-alpha (FIG. 2), and IL-6 (FIG. 3). In FIG. 4, it was demonstrated that fibroblasts augment the ability of minocycline to induce T regulatory cells.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.
1. Min, W. P., et al., Inhibitory feedback loop between tolerogenic dendritic cells and regulatory T cells in transplant tolerance. J Immunol, 2003. 170(3): p. 1304-12.
2. Zhang, X., et al., Generation of therapeutic dendritic cells and regulatory T cells for preventing allogeneic cardiac graft rejection. Clin Immunol, 2008. 127(3): p. 313-21.
3. Zhou, X., et al., Tolerogenic Vaccine Composited with Islet Derived Multipeptides and Cyclosporin A Induces pTreg and Prevents Type 1 Diabetes in Murine Model. Hum Vaccin Immunother, 2019.
4. Tabansky, I., et al., Targeting DEC-205(−)DCIR2(+) dendritic cells promotes immunological tolerance in proteolipid protein-induced experimental autoimmune encephalomyelitis. Mol Med, 2018. 24(1): p. 17.
5. Erlebacher, A., et al., Constraints in antigen presentation severely restrict T cell recognition of the allogeneic fetus. J Clin Invest, 2007. 117(5): p. 1399-411.
6. Chen, T., et al., Self-specific memory regulatory T cells protect embryos at implantation in mice. J Immunol, 2013. 191(5): p. 2273-81.
7. Harimoto, H., et al., Inactivation of tumor-specific CD8(+) CTLs by tumor-infiltrating tolerogenic dendritic cells. Immunol Cell Biol, 2013. 91(9): p. 545-55.
8. Ney, J. T., et al., Autochthonous liver tumors induce systemic T cell tolerance associated with T cell receptor down-modulation. Hepatology, 2009. 49(2): p. 471-81.
9. Cheung, A. F., et al., Regulated expression of a tumor-associated antigen reveals multiple levels of T-cell tolerance in a mouse model of lung cancer. Cancer Res, 2008. 68(22): p. 9459-68.
10. Bal, A., et al., Rapid tolerization of virus-activated tumor-specific CD8+ T cells in prostate tumors of TRAMP mice. Proc Natl Acad Sci USA, 2008. 105(35): p. 13003-8.
11. Jacobs, J. F., et al., Regulatory T cells in melanoma: the final hurdle towards effective immunotherapy? Lancet Oncol, 2012. 13(1): p. e32-42.
12. Pedroza-Gonzalez, A., et al., Activated tumor-infiltrating CD4+ regulatory T cells restrain antitumor immunity in patients with primary or metastatic liver cancer. Hepatology, 2013. 57(1): p. 183-94.
13. Donkor, M. K., et al., T cell surveillance of oncogene-induced prostate cancer is impeded by T cell-derived TGF-beta1 cytokine. Immunity, 2011. 35(1): p. 123-34.
14. Whiteside, T. L., Down-regulation of zeta-chain expression in T cells: a biomarker of prognosis in cancer? Cancer Immunol Immunother, 2004. 53(10): p. 865-78.
15. Whiteside, T. L., Signaling defects in T lymphocytes of patients with malignancy. Cancer Immunol Immunother, 1999. 48(7): p. 346-52.
16. Reichert, T. E., et al., Signaling abnormalities, apoptosis, and reduced proliferation of circulating and tumor-infiltrating lymphocytes in patients with oral carcinoma. Clin Cancer Res, 2002. 8(10): p. 3137-45.
17. Faria, A. M. and H. L. Weiner, Oral tolerance. Immunol Rev, 2005. 206: p. 232-59.
18. Weiner, H. L., Induction and mechanism of action of transforming growth factor-beta-secreting Th3 regulatory cells. Immunol Rev, 2001. 182: p. 207-14.
19. Fukaura, H., et al., Induction of circulating myelin basic protein and proteolipid protein-specific transforming growth factor-beta1-secreting Th3 T cells by oral administration of myelin in multiple sclerosis patients. J Clin Invest, 1996. 98(1): p. 70-7.
20. Palomares, O., et al., Induction and maintenance of allergen-specific FOXP3+ Treg cells in human tonsils as potential first-line organs of oral tolerance. J Allergy Clin Immunol, 2012. 129(2): p. 510-20, 520 el-9.
21. Yamashita, H., et al., Overcoming food allergy through acquired tolerance conferred by transfer of Tregs in a murine model. Allergy, 2012. 67(2): p. 201-9.
22. Park, K. S., et al., Type II collagen oral tolerance; mechanism and role in collagen-induced arthritis and rheumatoid arthritis. Mod Rheumatol, 2009.
23. Womer, K. L., et al., A pilot study on the immunological effects of oral administration of donor major histocompatibility complex class II peptides in renal transplant recipients. Clin Transplant, 2008. 22(6): p. 754-9.
24. Faria, A. M. and H. L. Weiner, Oral tolerance: therapeutic implications for autoimmune diseases. Clin Dev Immunol, 2006. 13(2-4): p. 143-57.
25. Park, M. J., et al., A distinct tolerogenic subset of splenic IDO(+)CD11b(+) dendritic cells from orally tolerized mice is responsible for induction of systemic immune tolerance and suppression of collagen-induced arthritis. Cell Immunol, 2012. 278(1-2): p. 45-54.
26. Thompson, H. S., et al., Suppression of collagen induced arthritis by oral administration of type II collagen: changes in immune and arthritic responses mediated by active peripheral suppression. Autoimmunity, 1993. 16(3): p. 189-99.
27. Song, F., et al., The thymus plays a role in oral tolerance induction in experimental autoimmune encephalomyelitis. Ann N Y Acad Sci, 2004. 1029: p. 402-4.
28. Hanninen, A. and L. C. Harrison, Mucosal tolerance to prevent type 1 diabetes: can the outcome be improved in humans? Rev Diabet Stud, 2004. 1(3): p. 113-21.
29. Wei, W., et al., A multicenter, double-blind, randomized, controlled phase III clinical trial of chicken type II collagen in rheumatoid arthritis. Arthritis Res Ther, 2009. 11(6): p. R180.
30. Thurau, S. R., et al., Molecular mimicry as a therapeutic approach for an autoimmune disease: oral treatment of uveitis patients with an MHC peptide crossreactive with autoantigen—first results. Immunol Lett, 1997. 57(1-3): p. 193-201.
31. Weiner, H. L., et al., Double-blind pilot trial of oral tolerization with myelin antigens in multiple sclerosis. Science, 1993. 259(5099): p. 1321-4.
32. Tikka, T. M. and J. E. Koistinaho, Minocycline provides neuroprotection against N-methyl-D-aspartate neurotoxicity by inhibiting microglia. J Immunol, 2001. 166(12): p. 7527-33.
33. Tikka, T., et al., Minocycline, a tetracycline derivative, is neuroprotective against excitotoxicity by inhibiting activation and proliferation of microglia. J Neurosci, 2001. 21(8): p. 2580-8.
34. He, Y., S. Appel, and W. Le, Minocycline inhibits microglial activation and protects nigral cells after 6-hydroxydopamine injection into mouse striatum. Brain Res, 2001. 909(1-2): p. 187-93.
35. Zhang, S. C., B. D. Goetz, and I. D. Duncan, Suppression of activated microglia promotes survival and function of transplanted oligodendroglial progenitors. Glia, 2003. 41(2): p. 191-8.
36. Dommergues, M. A., et al., Early microglial activation following neonatal excitotoxic brain damage in mice: a potential target for neuroprotection. Neuroscience, 2003. 121(3): p. 619-28.
37. Stirling, D. P., et al., Minocycline treatment reduces delayed oligodendrocyte death, attenuates axonal dieback, and improves functional outcome after spinal cord injury. J Neurosci, 2004. 24(9): p. 2182-90.
38. Kim, S. S., et al., Inhibitory action of minocycline on lipopolysaccharide-induced release of nitric oxide and prostaglandin E2 in BV2 microglial cells. Arch Pharm Res, 2004. 27(3): p. 314-8.
39. Tomas-Camardiel, M., et al., Minocycline reduces the lipopolysaccharide-induced inflammatory reaction, peroxynitrite-mediated nitration of proteins, disruption of the blood-brain barrier, and damage in the nigral dopaminergic system. Neurobiol Dis, 2004. 16(1): p. 190-201.
40. Hunter, C. L., et al., Minocycline protects basal forebrain cholinergic neurons from mu p75-saporin immunotoxic lesioning. Eur J Neurosci, 2004. 19(12): p. 3305-16.
41. Wang, Q., M. J. Rowan, and R. Anwyl, Beta-amyloid-mediated inhibition of NMDA receptor-dependent long-term potentiation induction involves activation of microglia and stimulation of inducible nitric oxide synthase and superoxide. J Neurosci, 2004. 24(27): p. 6049-56.
42. Suk, K., Minocycline suppresses hypoxic activation of rodent microglia in culture. Neurosci Lett, 2004. 366(2): p. 167-71.
43. Ryu, J. K., et al., Minocycline inhibits neuronal death and glial activation induced by beta-amyloid peptide in rat hippocampus. Glia, 2004. 48(1): p. 85-90.
44. Li, W. W., et al., Minocycline-mediated inhibition of microglia activation impairs oligodendrocyte progenitor cell responses and remyelination in a non-immune model of demyelination. J Neuroimmunol, 2005. 158(1-2): p. 58-66.
45. Giuliani, F., W. Hader, and V. W. Yong, Minocycline attenuates T cell and microglia activity to impair cytokine production in T cell-microglia interaction. J Leukoc Biol, 2005. 78(1): p. 135-43.
46. Ingham, E., Modulation of the proliferative response of murine thymocytes stimulated by IL-1, and enhancement of IL-1 beta secretion from mononuclear phagocytes by tetracyclines. J Antimicrob Chemother, 1990. 26(1): p. 61-70.
47. Kloppenburg, M., et al., The influence of tetracyclines on T cell activation. Clin Exp Immunol, 1995. 102(3): p. 635-41.
48. Kloppenburg, M., et al., The tetracycline derivative minocycline differentially affects cytokine production by monocytes and T lymphocytes. Antimicrob Agents Chemother, 1996. 40(4): p. 934-40.
49. Popovic, N., et al., Inhibition of autoimmune encephalomyelitis by a tetracycline. Ann Neurol, 2002. 51(2): p. 215-23.
50. Kalish, R. S. and S. Koujak, Minocycline inhibits antigen processing for presentation to human T cells: additive inhibition with chloroquine at therapeutic concentrations. Clin Immunol, 2004. 113(3): p. 270-7.
51. Zhang, Z. Y., et al., Improved outcome of EAN, an animal model of GBS, through amelioration of peripheral and central inflammation by minocycline. J Cell Mol Med, 2009. 13(2): p. 341-51.
52. Silveira, M. G., et al., Minocycline in the treatment of patients with primary sclerosing cholangitis: results of a pilot study. Am J Gastroenterol, 2009. 104(1): p. 83-8.
53. Nikodemova, M., et al., Minocycline attenuates experimental autoimmune encephalomyelitis in rats by reducing T cell infiltration into the spinal cord. J Neuroimmunol, 2010. 219(1-2): p. 33-7.
54. Szeto, G. L., et al., Minocycline attenuates HIV infection and reactivation by suppressing cellular activation in human CD4+ T cells. J Infect Dis, 2010. 201(8): p. 1132-40.
55. Maeda, M., et al., Suppressive efficacies of antimicrobial agents against human peripheral-blood mononuclear cells stimulated with T cell mitogen and bacterial superantigen. Arzneimittelforschung, 2010. 60(12): p. 760-8.
56. Szeto, G. L., et al., Minocycline suppresses activation of nuclear factor of activated T cells 1 (NFAT1) in human CD4+ T cells. J Biol Chem, 2011. 286(13): p. 11275-82.
57. Enose-Akahata, Y., et al., Minocycline modulates antigen-specific CTL activity through inactivation of mononuclear phagocytes in patients with HTLV-I associated neurologic disease. Retrovirology, 2012. 9: p. 16.
58. Michel-Monigadon, D., et al., Minocycline promotes long-term survival of neuronal transplant in the brain by inhibiting late microglial activation and T-cell recruitment. Transplantation, 2010. 89(7): p. 816-23.
59. Chen, M., et al., Minocycline inhibits caspase-1 and caspase-3 expression and delays mortality in a transgenic mouse model of Huntington disease. Nat Med, 2000. 6(7): p. 797-801.
60. Sanchez Mejia, R. O., et al., Minocycline reduces traumatic brain injury-mediated caspase-1 activation, tissue damage, and neurological dysfunction. Neurosurgery, 2001. 48(6): p. 1393-9; discussion 1399-401.
61. Du, Y., et al., Minocycline prevents nigrostriatal dopaminergic neurodegeneration in the MPTP model of Parkinson's disease. Proc Natl Acad Sci USA, 2001. 98(25): p. 14669-74.
62. Zhu, S., et al., Minocycline inhibits cytochrome c release and delays progression of amyotrophic lateral sclerosis in mice. Nature, 2002. 417(6884): p. 74-8.
63. Vincent, J. A. and S. Mohr, Inhibition of caspase-1/interleukin-1beta signaling prevents degeneration of retinal capillaries in diabetes and galactosemia. Diabetes, 2007. 56(1): p. 224-30.
64. Kim, H. S. and Y. H. Suh, Minocycline and neurodegenerative diseases. Behav Brain Res, 2009. 196(2): p. 168-79.
65. Kwon, Y. S., et al., Neuroprotective and antiepileptogenic effects of combination of anti-inflammatory drugs in the immature brain. J Neuroinflammation, 2013. 10: p. 30.
66. Levkovitch-Verbin, H., et al., Minocycline upregulates pro-survival genes and downregulates pro-apoptotic genes in experimental glaucoma. Graefes Arch Clin Exp Ophthalmol, 2014. 252(5): p. 761-72.
67. Wong, M. L., et al., Inflammasome signaling affects anxiety-and depressive-like behavior and gut microbiome composition. Mol Psychiatry, 2016. 21(6): p. 797-805.
68. Chen, W., et al., Activation of the TXNIP/NLRP3 inflammasome pathway contributes to inflammation in diabetic retinopathy: a novel inhibitory effect of minocycline. Inflamm Res, 2017. 66(2): p. 157-166.
69. Kim, N., et al., Minocycline promotes the generation of dendritic cells with regulatory properties. Oncotarget, 2016. 7(33): p. 52818-52831.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of increasing regenerative activity of a population of fibroblasts comprising subjecting said population with one or more agents capable of inhibiting NF-kappa B.

2. The method of claim 1, wherein said agent capable of inhibiting NF-kappa B suppresses ability of fibroblasts to produce one or more inflammatory cytokines.

3. The method of claim 2, wherein said inflammatory cytokine is TNF-alpha.

4. The method of claim 2, wherein said inflammatory cytokine is IL-1 beta.

5. The method of claim 2, wherein said inflammatory cytokine is IL-6.

6. The method of claim 1, wherein said inhibitor of NF-kappa B is minocycline.

7. The method of claim 1, wherein said inhibitor of NF-kappa B is administered together with an activator of AMPK to an individual in need thereof.

8. The method of claim 7, wherein said AMPK activator is metformin.

9. The method of claim 1, wherein said agent capable of inhibiting NF-kappa B is administered to an individual in need thereof prior to, and/or concurrent with, and/or subsequent to administration of said fibroblast.

10. The method of claim 1, wherein said fibroblast is obtained from a source that is either autologous, allogeneic, or xenogeneic with respect to an individual in need thereof.

11. The method of claim 1, wherein said inhibitor of NF-kappa B is selected from the group consisting of Oxytetracycline, Demeclocycline, Minocycline, Methacycline, Doxycycline, Chlortetracycline, Tetracycline, Sancycline, Chelocardin, 6-demethyl-6-deoxy-4-dedimethylaminotetracycline; tetracyclino-pyrazole; 7-chloro-4-dedimethylaminotetracycline; 4-hydroxy-4-dedimethylaminotetracycline; 12.alpha.-deoxy-4-dedimethylaminotetracycline; 5-hydroxy-6a-deoxy-4-dedimethylaminotetracycline; 4-dedimethylamino-12.alpha.-deoxyanhydrotetracycline; 7-dimethylamino-6-demethyl-6-deoxy-4-dedimethylaminotetracycline; tetracyclinonitrile; 4-oxo-4-dedimethylaminotetracycline 4,6-hemiketal; 4-oxO-11a C1-4-dedimethylaminotetracycline-4,6-hemiketal; 5a,6-anhydro-4-hydrazon-4-dedimethylamino tetracycline; 4-hydroxyimino-4-dedimethylaminotetracyclines; 4-hydroxyimino-4-dedimethylamino 5a,6-anhydrotetracyclines; 4-amino-4-dedimethylamino-5a, 6 anhydrotetracycline; 4-methylamino-4-dedimethylamino tetracycline; 4-hydrazono-11a-chloro-6-deoxy-6-demethyl-6-methylene-4-dedimethylamino tetracycline; tetracycline quaternary ammonium compounds; anhydrotetracycline betaines; 4-hydroxy-6-methyl pretetramides; 4-keto tetracyclines; 5-keto tetracyclines; 5a,11a dehydro tetracyclines; 11a C1-6, 12 hemiketal tetracyclines; 11a C1-6-methylene tetracyclines; 6, 13 diol tetracyclines; 6-benzylthiomethylene tetracyclines; 7,11a-dichloro-6-fluoro-methyl-6-deoxy tetracyclines; 6-fluoro (.alpha.)-6-demethyl-6-deoxy tetracyclines; 6-fluoro (.beta.)-6-demethyl-6-deoxy tetracyclines; 6-.alpha. acetoxy-6-demethyl tetracyclines; 6-.beta. acetoxy-6-demethyl tetracyclines; 7, 13-epithiotetracyclines; oxytetracyclines; pyrazolotetracyclines; 11a halogens of tetracyclines; 12a formyl and other esters of tetracyclines; 5, 12a esters of tetracyclines; 10, 12a-diesters of tetracyclines; isotetracycline; 12-a-deoxyanhydro tetracyclines; 6-demethyl-12a-deoxy-7-chloroanhydrotetracyclines; B-nortetracyclines; 7-methoxy-6-demethyl-6-deoxytetracyclines; 6-demethyl-6-deoxy-5a-epitetracyclines; 8-hydroxy-6-demethyl-6-deoxy tetracyclines; monardene; chromocycline; 5a methyl-6-demethyl-6-deoxy tetracyclines; 6-oxa tetracyclines, 6 thia tetracyclines, and a combination thereof.

12. The method of claim 1, wherein the population and the one or more agents are provided to an individual in need thereof, optionally in addition to one or more additional agents that enhance regenerative activity of said fibroblasts.

13. The method of claim 12, wherein said additional agent is selected from the group consisting of compounds that remove protein build up (e.g., geldanamycin), anti-inflammatory agents (e.g., glucocorticoids, non-steroidal anti-inflammatory drugs (e.g., ibuprofin, aspirin, etc.), omega-3 fatty acids (e.g., EPA, DHA, etc.), dexanabionol, etc.), compounds that increase energy available to cells (e.g., creatine, creatine phosphate, dichloroacetate, nicotinamide, riboflavin, carnitine, etc.), antioxidants (e.g., plant extracts (e.g., gingko biloba), co-enzyme Q-10, vitamin E (alpha-tocopherol), vitamin C (ascorbic acid), vitamin A (beta-carotene), selenium, lipoic acid, selegine, etc.), anti-glutamate therapies (e.g., remacemide, riluzole, lamotrigine, gabapentin, etc.), GABA-ergic therapies (e.g., baclofen, muscimol, etc.), gene transcription regulators (e.g., glucocorticoids, retinoic acid, etc.), erythropoietin, TNF-.alpha. antagonists, cholinesterase inhibitors, N-methyl-D-aspartate (NMDA) antagonists, opiod antagonists, neuronal membrane stabilizers (e.g., CDP-choline, etc.), calcium and sodium channel blockers, and prednisone.

14. The method of claim 1, wherein said fibroblasts are plastic-adherent.

15. The method of claim 1, wherein said fibroblasts express CD105.

16. The method of claim 1, wherein said fibroblasts express CD73.

17. A method of treating or preventing a neurological disorder in an individual, comprising the step of administering to an individual with a neurological disorder or at risk for a neurological disorder an effective amount of fibroblasts and one or more inhibitors of NFkappaB.

18. The method of claim 17, wherein the one or more inhibitors of NFkappaB are selected from the group consisting of Oxytetracycline, Demeclocycline, Minocycline, Methacycline, Doxycycline, Chlortetracycline, Tetracycline, Sancycline, Chelocardin, 6-demethyl-6-deoxy-4-dedimethylaminotetracycline; tetracyclino-pyrazole; 7-chloro-4-dedimethylaminotetracycline; 4-hydroxy-4-dedimethylaminotetracycline; 12.alpha.-deoxy-4-dedimethylaminotetracycline; 5-hydroxy-6a-deoxy-4-dedimethylaminotetracycline; 4-dedimethylamino-12.alpha.-deoxyanhydrotetracycline; 7-dimethylamino-6-demethyl-6-deoxy-4-dedimethylaminotetracycline; tetracyclinonitrile; 4-oxo-4-dedimethylaminotetracycline 4,6-hemiketal; 4-oxO-11a C1-4-dedimethylaminotetracycline-4,6-hemiketal; 5a,6-anhydro-4-hydrazon-4-dedimethylamino tetracycline; 4-hydroxyimino-4-dedimethylaminotetracyclines; 4-hydroxyimino-4-dedimethylamino 5a,6-anhydrotetracyclines; 4-amino-4-dedimethylamino-5a, 6 anhydrotetracycline; 4-methylamino-4-dedimethylamino tetracycline; 4-hydrazono-11a-chloro-6-deoxy-6-demethyl-6-methylene-4-dedimethylamino tetracycline; tetracycline quaternary ammonium compounds; anhydrotetracycline betaines; 4-hydroxy-6-methyl pretetramides; 4-keto tetracyclines; 5-keto tetracyclines; 5a,11a dehydro tetracyclines; 11a C1-6, 12 hemiketal tetracyclines; 11a C1-6-methylene tetracyclines; 6, 13 diol tetracyclines; 6-benzylthiomethylene tetracyclines; 7,11a-dichloro-6-fluoro-methyl-6-deoxy tetracyclines; 6-fluoro (.alpha.)-6-demethyl-6-deoxy tetracyclines; 6-fluoro (.beta.)-6-demethyl-6-deoxy tetracyclines; 6-.alpha. acetoxy-6-demethyl tetracyclines; 6-.beta. acetoxy-6-demethyl tetracyclines; 7, 13-epithiotetracyclines; oxytetracyclines; pyrazolotetracyclines; 11a halogens of tetracyclines; 12a formyl and other esters of tetracyclines; 5, 12a esters of tetracyclines; 10, 12a-diesters of tetracyclines; isotetracycline; 12-a-deoxyanhydro tetracyclines; 6-demethyl-12a-deoxy-7-chloroanhydrotetracyclines; B-nortetracyclines; 7-methoxy-6-demethyl-6-deoxytetracyclines; 6-demethyl-6-deoxy-5a-epitetracyclines; 8-hydroxy-6-demethyl-6-deoxy tetracyclines; monardene; chromocycline; 5a methyl-6-demethyl-6-deoxy tetracyclines; 6-oxa tetracyclines, 6 thia tetracyclines, and a combination thereof.

19. The method of claim 17, wherein the inhibitor of NFkappaB is minocycline or an analogue thereof.

20. The method of claim 17, wherein the fibroblasts are administered to the individual before, at the same time as, or after administration of the one or more inhibitors.

21. The method of claim 17, wherein the individual is a professional or recreational athlete or wherein the individual has a vocation at risk for head injury.

22. The method of claim 17, wherein the individual is further administered one or more agents selected from the group consisting of compounds that remove protein build up (e.g., geldanamycin), anti-inflammatory agents (e.g., glucocorticoids, non-steroidal anti-inflammatory drugs (e.g., ibuprofin, aspirin, etc.), omega-3 fatty acids (e.g., EPA, DHA, etc.), dexanabionol, etc.), compounds that increase energy available to cells (e.g., creatine, creatine phosphate, dichloroacetate, nicotinamide, riboflavin, carnitine, etc.), antioxidants (e.g., plant extracts (e.g., gingko biloba), co-enzyme Q-10, vitamin E (alpha-tocopherol), vitamin C (ascorbic acid), vitamin A (beta-carotene), selenium, lipoic acid, selegine, etc.), anti-glutamate therapies (e.g., remacemide, riluzole, lamotrigine, gabapentin, etc.), GABA-ergic therapies (e.g., baclofen, muscimol, etc.), gene transcription regulators (e.g., glucocorticoids, retinoic acid, etc.), erythropoietin, TNF-.alpha. antagonists, cholinesterase inhibitors, N-methyl-D-aspartate (NMDA) antagonists, opiod antagonists, neuronal membrane stabilizers (e.g., CDP-choline, etc.), calcium and sodium channel blockers, and prednisone.

23. The method of claim 17, wherein the neurological disorder is central nervous system injury, a chronic injury, an acute injury, or a disease.

24. The method of claim 23, wherein the chronic injury is chronic traumatic encephalopathy.

25. The method of claim 17, wherein the neurological disorder is Alzheimer's Disease (AD), age-associated memory impairment, mild cognitive impairment, cerebrovascular dementia. Acute Spinal Cord Injury, Amyotrophic Lateral Sclerosis (ALS), Ataxia, Bell's Palsy, Brain Tumors, Cerebral Aneurysm, Epilepsy, Seizures, Guillain-Barré Syndrome, Meningitis, Multiple Sclerosis, Muscular Dystrophy, Parkinson's Disease, migraine, stroke, encephalitis, Myasthenia Gravis, or a combination thereof.

26. A method of suppressing inflammation in an individual, comprising the step of providing to the individual a therapeutically effective amount of a population of fibroblasts and one or more inhibitors of NFkappaB.

27. The method of claim 26, wherein the one or more inhibitors of NFkappaB are selected from the group consisting of Oxytetracycline, Demeclocycline, Minocycline, Methacycline, Doxycycline, Chlortetracycline, Tetracycline, Sancycline, Chelocardin, 6-demethyl-6-deoxy-4-dedimethylaminotetracycline; tetracyclino-pyrazole; 7-chloro-4-dedimethylaminotetracycline; 4-hydroxy-4-dedimethylaminotetracycline; 12.alpha.-deoxy-4-dedimethylaminotetracycline; 5-hydroxy-6a-deoxy-4-dedimethylaminotetracycline; 4-dedimethylamino-12.alpha.-deoxyanhydrotetracycline; 7-dimethylamino-6-demethyl-6-deoxy-4-dedimethylaminotetracycline; tetracyclinonitrile; 4-oxo-4-dedimethylaminotetracycline 4,6-hemiketal; 4-oxO-11a C1-4-dedimethylaminotetracycline-4,6-hemiketal; 5a,6-anhydro-4-hydrazon-4-dedimethylamino tetracycline; 4-hydroxyimino-4-dedimethylaminotetracyclines; 4-hydroxyimino-4-dedimethylamino 5a,6-anhydrotetracyclines; 4-amino-4-dedimethylamino-5a, 6 anhydrotetracycline; 4-methylamino-4-dedimethylamino tetracycline; 4-hydrazono-11a-chloro-6-deoxy-6-demethyl-6-methylene-4-dedimethylamino tetracycline; tetracycline quaternary ammonium compounds; anhydrotetracycline betaines; 4-hydroxy-6-methyl pretetramides; 4-keto tetracyclines; 5-keto tetracyclines; 5a,11a dehydro tetracyclines; 11a C1-6, 12 hemiketal tetracyclines; 11a C1-6-methylene tetracyclines; 6, 13 diol tetracyclines; 6-benzylthiomethylene tetracyclines; 7,11a-dichloro-6-fluoro-methyl-6-deoxy tetracyclines; 6-fluoro (.alpha.)-6-demethyl-6-deoxy tetracyclines; 6-fluoro (.beta.)-6-demethyl-6-deoxy tetracyclines; 6-.alpha. acetoxy-6-demethyl tetracyclines; 6-.beta. acetoxy-6-demethyl tetracyclines; 7, 13-epithiotetracyclines; oxytetracyclines; pyrazolotetracyclines; 11a halogens of tetracyclines; 12a formyl and other esters of tetracyclines; 5, 12a esters of tetracyclines; 10, 12a-diesters of tetracyclines; isotetracycline; 12-a-deoxyanhydro tetracyclines; 6-demethyl-12a-deoxy-7-chloroanhydrotetracyclines; B-nortetracyclines; 7-methoxy-6-demethyl-6-deoxytetracyclines; 6-demethyl-6-deoxy-5a-epitetracyclines; 8-hydroxy-6-demethyl-6-deoxy tetracyclines; monardene; chromocycline; 5a methyl-6-demethyl-6-deoxy tetracyclines; 6-oxa tetracyclines, 6 thia tetracyclines, and a combination thereof.

28. The method of claim 26, wherein the inhibitor of NFkappaB is minocycline.
